(19) United States
(12) Patent Application Publication
(10) Pub. No.: US 2012/0029638 A1
(43) Pub. Date: Feb. 2, 2012

(54) VERTEBRAL BODY REPLACEMENT DEVICE CONFIGURED TO DELIVER A THERAPEUTIC SUBSTANCE

(75) Inventors: Keith E. Miller, Germantown, TN (US); Jonathan E. Blackwell, Arlington, TN (US); Rajesh Remesh, Memphis, TN (US)

(73) Assignee: WARSAW ORTHOPEDIC, INC., Warsaw, IN (US)

(21) Appl. No.: 12/847,069

(22) Filed: Jul. 30, 2010

(51) Int. Cl. A61F 2/44

(52) U.S. Cl. 623/17.12; 623/17.11

(57) ABSTRACT

Embodiments of the invention include a medical implant having a vertebral body replacement device, alone or in combination with other complimentary elements, configured to deliver one or more therapeutic substances. The one or more therapeutic substances may be contained in the medical implant or be ported to the medical implant.

### FIELD OF THE INVENTION

The present invention relates generally to the field of medical implants, and more particularly relates to medical implants that include a vertebral body replacement device that alone, or in combination with other components, are configured to deliver a therapeutic substance.

### BACKGROUND

The use of therapeutic substances in combination with medical implants has beneficial characteristics in many treatments. Therapeutic substances may be useful in promoting healing, fighting infection and disease by killing various pathogens such as bacteria, viruses, and microorganisms, promoting favorable cellular activity, killing cancer cells, or any of a wide variety of beneficial results. It may be advantageous to associate a therapeutic substance with a medical implant where the medical implant is implanted in a particularly advantageous location for effective application of the therapeutic substance.

It is a continuing challenge in the art to provide medical implants that may be conveniently and securely placed to deliver effective amounts of therapeutic substances in effective locations. A procedure for which delivery of therapeutic substances may be useful is a vertebral body replacement procedure. It is sometimes necessary to remove one or more vertebrae, or a portion of the vertebrae, from the human spine in response to various pathologies. For example, one or more of the vertebrae may become damaged as a result of tumor growth, or may become damaged by a traumatic or other event. Removal, or excision, of a vertebra may be referred to as a vertebrectomy. Excision of a generally anterior portion, or vertebral body, of the vertebra may be referred to as a corpectomy. An implant is usually placed between the remaining vertebrae to provide structural support for the spine as a part of a corpectomy or vertebrectomy. FIG. 1 illustrates four vertebrae, V₁-V₄ of a typical lumbar spine and three spinal discs, D₁-D₃. As illustrated, V₃ is a damaged vertebra and all or a part of V₃ could be removed to help stabilize the spine. If removed along with spinal discs D₂ and D₃, an implant may be placed between vertebrae V₂ and V₄. In some cases, the implant inserted between the vertebrae is designed to facilitate fusion between remaining vertebrae. In other cases, especially when treating tumors, the ultimate goal of the procedure is spinal stability, regardless of fusion. A successful procedure may decrease pain, preserve or enhance neurological function and allow a patient greater mobility without an external orthosis. Sometimes an implant is designed to replace the function of the excised vertebra and discs. All or part of more than one vertebra may be damaged and require removal and replacement in some circumstances. If only a portion of a vertebral body and adjacent discs are removed and replaced, the procedure is called a hemi-vertebrectomy. Corpectomy, vertebrectomy, hemi-vertebrectomy or any other full or partial vertebral body excision may all be referred to herein as vertebral body replacement.

Improved devices may be configured to provide delivery of one or more therapeutic substances from vertebral body replacement devices or from a component associated with vertebral body replacement devices. It may be advantageous to provide medical implants capable of delivering a therapeutic substance that may be placed in a sequence that is complementary to existing surgical procedures.

### SUMMARY

An embodiment of the invention is a medical implant configured to deliver a therapeutic substance. The medical implant embodiment may include a vertebral body replacement device having a first end and a substantially opposite second end, and a sheath coupled to the vertebral body replacement device. The sheath may cover substantially all of the vertebral body replacement device from the first end to the second end. The sheath may also include a therapeutic substance deliverable from the sheath to one or more anatomic structures positionable adjacent to the medical implant.

Another embodiment of the invention is a medical implant configured to deliver a therapeutic substance. The medical implant embodiment may include at least one therapeutic substance reservoir, two or more nozzles in fluid communication with the at least one therapeutic substance reservoir, and a vertebral body replacement device. The vertebral body replacement device may have a first end, a substantially opposite second end, and one or more sides extending from the first end to the second end, wherein the one or more sides at least in part define an interior of the vertebral body replacement device. The two or more nozzles may be coupled to the vertebral body replacement device to direct therapeutic substance from the interior of the vertebral body replacement device.

Yet another embodiment of the invention is a medical implant configured to deliver a therapeutic substance. The medical implant embodiment may include a vertebral body replacement device having a first end, a substantially opposite second end, and one or more sides extending from the first end to the second end, wherein the one or more sides at least in part define an interior of the vertebral body replacement device. The medical implant embodiment may also include an insert captured within the interior of the vertebral body replacement device. The insert may include one or more therapeutic substances deliverable from the insert and a release mechanism for permitting the one or more therapeutic substances to be delivered from the insert.

### BRIEF DESCRIPTION OF THE DRAWINGS

FIG. 1 is an elevation view of a segment of a lumbar spine.

FIG. 2 is a perspective view of an embodiment of a medical implant configured to deliver a therapeutic substance.

FIG. 3 is a cross-sectional view of an embodiment of a medical implant configured to deliver a therapeutic substance in a contracted state.

FIG. 4 is a cross-sectional perspective view of the medical implant of FIG. 3 from a transverse direction and in an expanded state.

FIG. 5 is a top, plan view of an embodiment of a medical implant configured to deliver a therapeutic substance next to a spinal cord.

FIG. 6 is a cross-sectional perspective view of an embodiment of a medical implant configured to deliver a therapeutic substance.

FIG. 7 is a perspective view of an embodiment of a medical implant configured to deliver a therapeutic substance illustrating two alternative inserts.

FIG. 8 is a cross-sectional view of an embodiment of an insert of the medical implant of FIG. 7.

FIG. 9 is a cross-sectional view of an embodiment of an insert of the medical implant of FIG. 7.

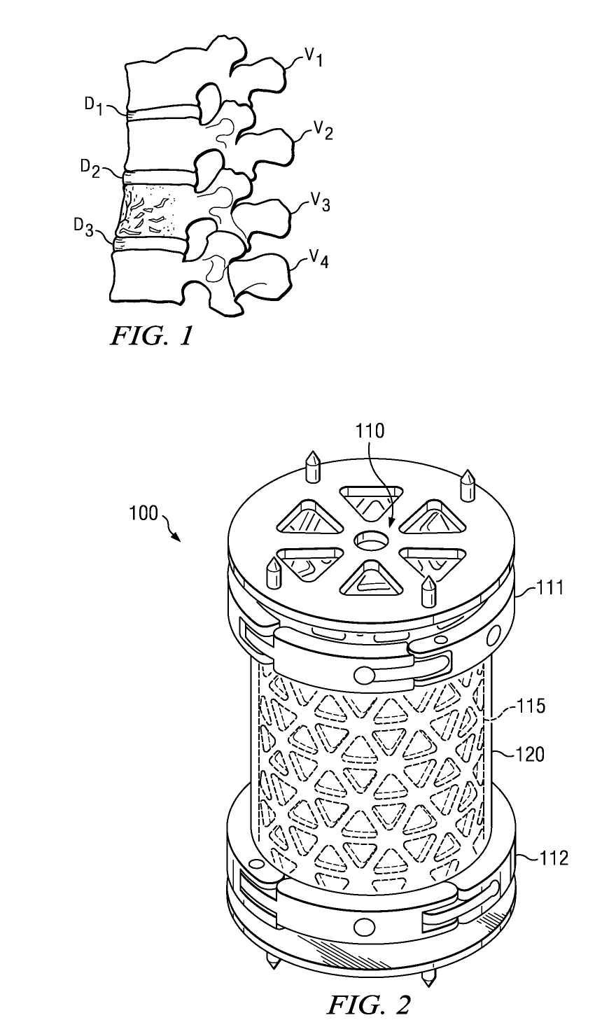

### DETAILED DESCRIPTION

An embodiment of a medical implant configured to deliver a therapeutic substance is shown in FIG. 2. The illustrated medical implant 100 includes a vertebral body replacement device 110 having a first end 111 and a substantially opposite second end 112. The medical implant 100 includes a section of PYRAMESH® Surgical Titanium Mesh between two SCEPTOR™ Universal Endcleats, all available from Medtronic, Inc. The mesh, or strut 115, illustrated includes generally triangular openings. In other embodiments, a mesh or other strut may include holes of any shape, slots, or may not include openings. In addition or as an alternative to titanium, the mesh or other strut may be made from any biocompatible material. For example and without limitation, the mesh or other strut may be made in whole or in part from one or more of polyetheretherketone (PEEK), cobalt chrome, stainless steel, or any biocompatible metal, metal alloy, polymer or a bone or bone-based material such as allograft, xenograft, demineralized bone, and autograft.

In the illustrated embodiment, the first end 111 and the second end 112 incorporate SCEPTOR™ Universal Endcleats. In some embodiments, the vertebral body replacement device may include other devices or structures at one or both of its ends or may be a unitary device that has no other separate structures or devices associated with its ends. The illustrated vertebral body replacement device 110 has a substantially circular cross-sectional shape. In other embodiments, a vertebral body replacement device may have a lateral periphery of one or more walls having a cross-sectional shape that is substantially oval, kidney shape, triangle, rectangle, square, any polygonal or curved shape, or any combination of shapes.

A sheath 120 is shown coupled to the vertebral body replacement device 110 and covering substantially all of the vertebral body replacement device 110 from the first end 111 to the second end 112. The illustrated sheath 120 terminates outside of each of the SCEPTOR™ Universal Endcleats. In other embodiments, a sheath may be captured within endcleats or other end pieces or may extend around a periphery of an end piece of any functional type.

The illustrated sheath 120 includes a therapeutic substance deliverable from the sheath 120 to one or more anatomic structures positionable adjacent to the medical implant 100. The delivery of therapeutic substance may be in any direction from a medical implant. The amount and timing of delivery of therapeutic substance may be controlled by one or more of placement of the sheath, inclusion of type and amount of therapeutic substance in various parts of the sheath, and release characteristics of the therapeutic substance or the sheath.

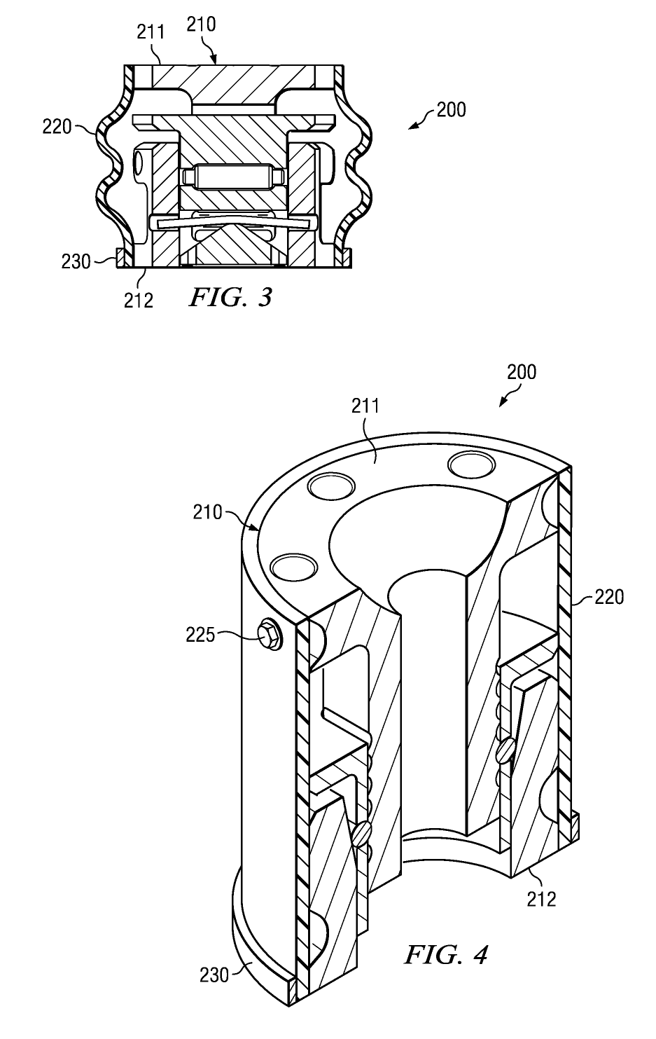

Another embodiment of a medical implant configured to deliver a therapeutic substance is shown in FIGS. 3 and 4. The illustrated medical implant 200 includes a vertebral body replacement device 210 having a first end 211 and a substantially opposite second end 212. The vertebral body replacement device 210 is an expandable device configured to be expanded from a first state, where the first end 211 and the second end 212 are a distance apart, as illustrated, for example, in FIG. 3. From this first state, the vertebral body replacement device 210 may be expanded to a second state where the first end 211 and the second end 212 are a greater distance apart. An example second state is illustrated in FIG. 4. The vertebral body replacement device 210 shown is a T2 XVBR™ Spinal Implant available from Medtronic, Inc. The vertebral body replacement device 210 may include titanium material and may be expandable by ratcheting or sliding and locking between components of the first and second ends. In addition or as an alternative to titanium, the vertebral body replacement device 210 may be made from any biocompatible material. For example and without limitation, the vertebral body replacement device 210 may be made in whole or in part from polyetheretherketone (PEEK), cobalt chrome, stainless steel, and any biocompatible metal, metal alloy, polymer, or a bone or bone-based material such as one or more of allograft, xenograft, demineralized bone, and autograft.

The medical implant 200 may also include additional pieces such as cleats or other connection mechanisms to extend the length of the device or provide connection to adjacent vertebrae at one or both of the first and second ends 211, 212. Similar to the vertebral body replacement device 110, the vertebral body replacement device 210 may include any functional shape or configuration.

A sheath 220 is shown coupled to the vertebral body replacement device 210 and covering substantially all of the vertebral body replacement device 210 from the first end 211 to the second end 212. The sheath 220 is shown coupled to the vertebral body replacement device 210 by a sheath fastener 225 at a first location near the first end 211. The sheath 220 is also shown coupled to the vertebral body replacement device 210 by a band 230 at a second location near the second end 212. The illustrated sheath fastener 225 is a bolt or screw and washer. In other embodiments, any other screw, pin, clip, or other effective mechanism may be used. A separate washer may be used, a washer may not be used, or fasteners of some embodiments may incorporate a washer or force spreading portion in a unitary device. The illustrated band 230 for fastening a sheath wraps around the second end 212 and is shortened or tightened to capture the sheath 220 between the band 230 and the second end 212. In some embodiments, the band 230 may be a hose clap of any variety or any device effective to wrap around or otherwise capture the sheath 220 relative to a vertebral body replacement device.

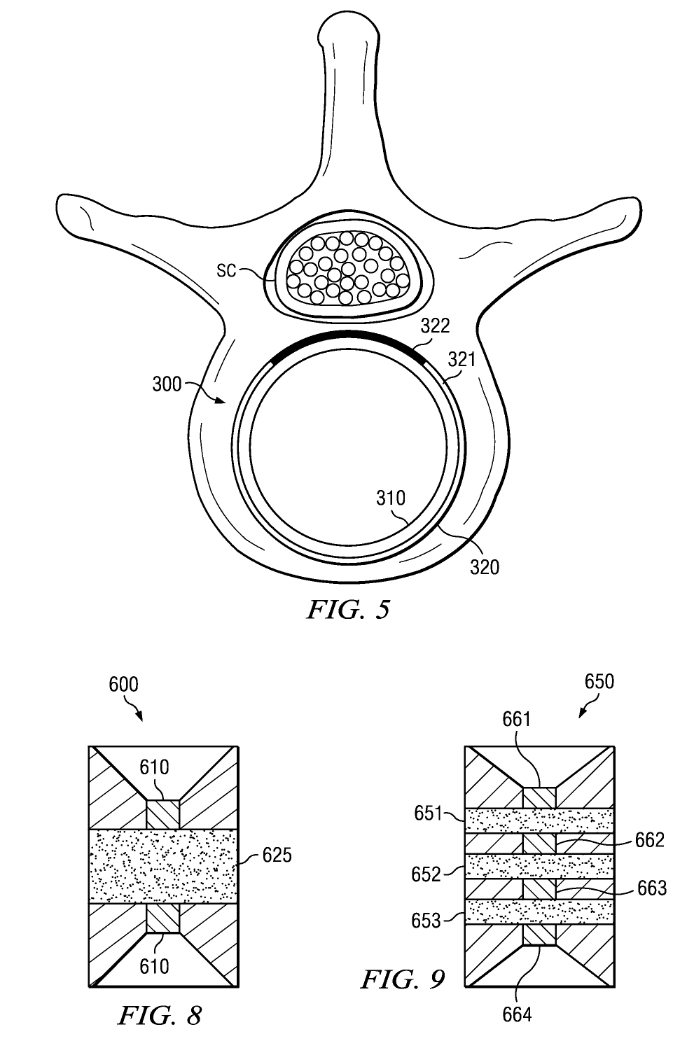

Another embodiment of a medical implant configured to deliver a therapeutic substance is shown in FIG. 5 in cross-section. The illustrated medical implant 300 includes a vertebral body replacement device 310. The vertebral body replacement device 310 may be of any functional size or shape. A sheath 320 is shown coupled to the vertebral body replacement device 310. The medical implant 300 is depicted next to a spinal cord (SC). The sheath 320 is illustrated with a therapeutic region 321 and a non-therapeutic region 322. The non-therapeutic region 322 is shown implanted generally adjacent to the spinal cord and other major neural structures in the embodiment of FIG. 5. In other embodiments, two or more regions of a sheath may include differing therapeutics or no therapeutics. Non-therapeutic regions or regions containing particular therapeutic substances may be placed next to any area helpful in achieving a desired clinical result, including but not limited to, anterior, posterior, oblique, lateral, medial, caudal, and cephalad.

Any of the sheaths disclosed specifically herein (120, 220, 320) or otherwise represented by this disclosure may include a therapeutic substance deliverable from the sheath to one or more anatomic structures positionable adjacent to the medical implant of which a sheath is a part or to which a sheath may be added. The delivery of therapeutic substance may be in any direction from a medical implant. The amount and timing of delivery of therapeutic substances may be controlled by one or more of placement of the sheath, inclusion of type and amount of therapeutic substance in various parts of the sheath, and release characteristics of the sheath and the therapeutic substances. In some embodiments, the sheath may be made at least in part from a relatively rigid material. In other embodiments, the sheath may be made at least in part from a relatively elastic material or include relatively elastic regions. A sheath that includes elastic material or regions may be capable of stretching over a component to be affixed to the component, or may, for example, stretch to remain in place over an expandable vertebral body to move from a collapsed state to an expanded state. A sheath may include one or more specific stretching portions or may be formed to include folds or bellows that expand when a force is applied.

In the embodiments shown in FIGS. 2-5, each sheath includes a therapeutic substance deliverable from the sheath. A sheath may be configured to release at least a portion of the therapeutic substance when the sheath is exposed to an at least in part aqueous substance. The aqueous substances of some embodiments are bodily fluids. Release may be accomplished through an activated release via a pH change or other local environmental change. The local environmental change may result from physiological changes, be induced by injecting a separate agent, or be caused by another mechanism. Release may also be induced in response to electro-magnetic energy directed toward a sheath. Embodiments of the sheath in whole or in part may be constructed of biocompatible materials of various types. Example materials include, but are not limited to, non-reinforced polymers, reinforced polymer composites, metals, ceramics and combinations thereof. In some embodiments, the sheath may be constructed of sections of bone or other tissues. Tissue materials include, but are not limited to, autograft, allograft, or xenograft, and may be resorbable or non-resorbable in nature. Examples of other tissue materials include hard tissues, connective tissues, demineralized bone matrix, and combinations thereof.

All or a part of a sheath may include a polymeric body configured to elute the therapeutic substance. The polymeric body may further elute the therapeutic substance at a predetermined rate. Alternatively or in addition, a sheath may at least in part be porous, and the therapeutic substance may be at least in part disposed in the pores of a sheath.

The sheath in whole or in part may comprise a polymeric material into or onto which a therapeutic substance is incorporated. Any polymeric material may be used. The polymeric material may be biocompatible and capable of presenting or eluting the therapeutic substance in an effective amount. Biocompatible polymers may be obtained from natural or synthetic sources, and may be bioresorbable. Examples of natural materials of which the polymer may be composed include collagen, elastin, silk, and demineralized bone matrix. Other examples of suitable polymeric materials include organic polymers such as silicones, polyamines, polystyrene, polyurethane, acrylates, polysilanes, polysulfone, methoxysilanes, and the like. Other polymers that may be utilized include polyolefins, polyisobutylene and ethylene-alphaolefin copolymers; acrylic polymers and copolymers, ethylene-covinylacetate, polybutylmethacrylate; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; carboxymethyl cellulose; polyphenyleneoxide; polytetrafluoroethylene (PTFE); polyethylene, low density polyethylene; polymethylmethacrylate (PMMA); polyetheretherketone (PEEK); and polyetherketoneketone (PEKK). The polymer may also be a polymeric hydroxyethylmethacrylate (PHEMA). Suitable bioresorbable synthetic polymers include poly(L-lactide), poly(D,L-lactide), poly(L-co-D,L-lactide), polyglycolide, poly(lactide-co-glycolide), poly(hydroxylbutyrate), poly(hydroxyvalerate), tyrosine-derived polycarbonate, polyanhydride, polyorthoester, polyphosphazene, poly(dioxanone), and polyglyconate. Other similar polymers known to the art may be used and various mixtures of polymers may be combined to adjust the properties of the composition as desired.

A therapeutic substance may be incorporated into or coated on a polymeric material of a sheath using any known or later developed technique. For example, the therapeutic substance may be adhered to a surface of any part of a sheath, adsorbed into a sheath, or compounded into the polymeric material that forms a sheath. Accordingly, the therapeutic substance may be embedded, coated, mixed or dispersed on or in the material of a sheath. A coating method may be determined by the material of a sheath and the therapeutic substance utilized. Such methods include but are not limited to, dipping, spraying, rolling, plating and embedding the coating into a surface by any means. For example, a polymeric sheath may be coated by dipping or spray coating polymeric resin and crosslinker with the therapeutic substance as substituent or dissolved within the polymer. Curing may be achieved chemically, photochemically or thermally. Other common methods include dip or spray coating water insoluble resin containing a therapeutic substance followed by drying or grafting directly onto the substrate chemically or photochemically.

Additional examples of ways to form at least a portion of a sheath include blending a therapeutic substance with a polymer and then forming the polymer into the sheath, or portion of the sheath. Alternatively, the therapeutic substance may be in a solution with the polymer to form a coating. The therapeutic substance may be attached to a polymeric material by a chemical modification of the surface such as surface grafting by hydrolyzable linkage of the therapeutic substance to the surface or by photolinking the therapeutic substance to the surface. Surface polymerization, derivatization or absorption may also be used. Other examples of obtaining a surface bound therapeutic substance include any existing means, such as ion implantation, chemical modification of the surface, photochemical or chemical grafting or formation of a crosslinked surface immobilized network. Silver ions, where used, may be deposited on a surface of a sheath by vacuum deposition, ion sputtering or surface deposition, among others. A surface of a sheath may be pretreated according to known methods such as plasma treatment prior to exposure to the coating material. Where solvents are present in a therapeutic substance, such solvents may be biocompatible if residue remains after the therapeutic substance is applied.

A therapeutic substance may comprise one or more of the following: antibiotics, antiseptics, analgesics, bone growth promoting substances, anti-inflammatants, anti-coagulants, antifungal agents, steroids, enzymes, immunosuppressants, antithrombogenic compositions, vaccines, hormones, growth inhibitors, growth stimulators, chemotherapy drugs, and the like. A therapeutic substance may be any drug or bioactive agent which can serve a useful therapeutic or even diagnostic function when released into a patient. More than one therapeutic substance may be present in or on a sheath for a particular treatment within the scope of the invention.

A therapeutic substance may include pharmaceuticals that target particular cells, such as but not limited to, cancer cells. A therapeutic substance may be any radiopharmaceutical or radionuclide, for example. A therapeutic substance may be a pure bone-seeking radioisotopes, such as strontium-89 and phosphorus-32 or a radioisotope that has been combined with other bone-seeking agents, such as samarium-153, rhenium-186, and iodine-131.

A therapeutic substance may include a DNA-damaging agent, such as chlorambucil, cyclophosphamide or melphalan, collectively referred to as alkylating agents. These DNA-damaging agents damage the DNA so severely that the cancer cell is killed. Other DNA-damaging agents, such as carboplatinum, attach to the DNA and prevent the cancer cell from growing.

A therapeutic substance may include antitumor antibiotics, such as daunorubicin, doxorubicin, idarubicin, and mitoxantrone, which insert themselves into the DNA of a cancer cell, prevent the DNA from functioning normally, and often kill the cancer cell.

A therapeutic substance may include antimetabolites, such as methotrexate, fludarabine, and cytarabine. These drugs mimic substances that the cancer cell needs to build DNA and RNA. When a cancer cell uses the antimetabolite instead of the natural substances, it cannot produce normal DNA or RNA, and the cell dies.

A therapeutic substance may include DNA-repair enzyme inhibitors, such as etoposide or topotecan. These inhibitors attack the cancer cell proteins that normally repair any damage to the cell DNA. Repair of DNA damage is a normal and vital process in the cell, without which the cancer cell is much more susceptible to damage and is prevented from growing.

A therapeutic substance may include vincristine or vinblastine. These agents damage cancer cells by blocking mitosis. This prevents the cancer cells from dividing and multiplying.

A therapeutic substance may include antibodies that are made specifically to attach to cancer cells. Once these antibodies attach to the cancer cells, the antibodies interfere with the cells' functions and kill the cells. Some antibodies may also be linked to a toxin or radioactive substance. When these antibodies attach to cancer cells, one or more of the antibodies, the toxin, and the radioactive substance work to kill cancer cells.

A therapeutic substance may include radiation sources that remain physically within a sheath, but that emit radiation in a desired direction from the sheath. By way of non-limiting example, a radiation source may be a radionuclide, such as I-125, I-131, Yb-169, Ir-192 or other radionuclides that emit photons, beta particles, gamma radiation, or other therapeutic energy or substances. A radioactive therapeutic substance may also be a fluid made from any solution of radionuclide(s), e.g., a solution of I-125 or I-131, or a radioactive mixture may be produced using a slurry of a suitable fluid containing small particles of solid radionuclides, such as Au-198, Y-90. Radionuclides may also be provided in a gel. One radioactive material useful in some embodiments is Iotrex®, a nontoxic, water soluble, nonpyrogenic solution containing sodium 3-(125I)iodo-4-hydroxybenzenesulfonate (125I-HBS), available from Proxima Therapeutics, Inc. of Alpharetta, Ga. Radioactive micro spheres of the type available from the 3M Company of St. Paul, Minn., may also be incorporated into or introduced into a sheath. A radioactive source may be preloaded into a medical implant at the time of manufacture or loaded after the medical implant has been implanted.

A therapeutic substance may include any antibiotic suitable for use in a human. As used herein, “antibiotic” means an antibacterial agent. The antibacterial agent may have bateriostatic and/or bacteriocidal activities. Nonlimiting examples of classes of antibiotics that may be used include tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sufonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole) and beta-lactam inhibitors (e.g. sulbactam). Nonlimiting examples of specific antibiotics that may be used include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other antibiotics may also be used.

To enhance the likelihood that bacteria will be killed or inhibited, it may be desirable to combine one or more antibiotics. It may also be desirable to combine one or more antibiotics with one or more antiseptics. Agents having different mechanisms of action and/or different spectrums of action may be most effective in achieving such an effect.

Any antiseptic suitable for use in a human may be used as or as part of a therapeutic substance. As used herein, “antiseptic” means an agent capable of killing or inhibiting the growth of one or more of bacteria, fungi, or viruses. Antiseptic includes disinfectants. Nonlimiting examples of antiseptics include hexachlorophene, cationic bisiguanides (i.e. chlorhexidine, cyclohexidine) iodine and iodophores (i.e. povidone-iodine), para-chloro-meta-xylenol, triclosan, furan medical preparations (i.e. nitrofurantoin, nitrofurazone), methenamine, aldehydes (glutaraldehyde, formaldehyde), silver sulfadiazine and alcohols. It may be desirable that the one or more antiseptics selected kill or inhibit the growth of one or more microbes that are associated with infection following surgical implantation of a medical device. Such bacteria may include Stapholcoccus aureus, Staphlococcus epidermis, Pseudomonus auruginosa, and Candidia. To enhance the likelihood that microbes will be killed or inhibited, it may be desirable to combine one or more antiseptics. It may also be desirable to combine one or more antiseptics with one or more antibiotics. Antimicrobial agents having different mechanisms of action and/or different spectrums of action may be most effective in achieving such an effect.

A therapeutic substance may be present in the sheath at any concentration effective, either alone or in combination with another therapeutic substance. Generally, a therapeutic substance may be present in a sheath at a range of between about 0.5% and about 20% by weight. For example, a therapeutic substance may be present in a sheath at a range of between about 0.5% and about 15% by weight or between about 0.5% and about 10% by weight.

A therapeutic substance may comprise an antimicrobial material including metals known to have antimicrobial properties, such as silver, gold, platinum, palladium, iridium, tin, copper, antimony, bismuth, selenium and zinc. Compounds of these metals, alloys containing one or more of these metals, or salts of these metals may be coated onto the surface of a sheath or added to the material from which a sheath is made during the manufacture of the sheath or compounded into the base material. One therapeutic substance will contain silver ions and may be obtained through the use of silver salts, such as silver acetate, silver benzoate, silver carbonate, silver iodate, silver iodide, silver lactate, silver laurate, silver nitrate, silver oxide, silver palmitate, silver protein, or silver sulfadiazine, among others. In an embodiment where selenium is used, the selenium may be bonded to the surface of a sheath, providing an antimicrobial coating.

A therapeutic substance may also comprise an osteoconductive, osteogenic, or osteoinductive material. For example and without limitation, a therapeutic substance may include various bioceramic materials, calcium phosphate and other members of the calcium phosphate family, fluorapatite, bioactive glass, and collagen-based materials. Members of the calcium phosphate family include materials such as hydroxyapatite, α-tricalcium phosphate, β-tricalcium phosphate, tetracalcium phosphate, dicalcium phosphate dihydrate, ocatacalcium phosphate, and the like. A therapeutic substance may include an osteoinductive or osteogenic materials such as osteoblast cells, platelet-derived growth factors (PDGFs), bone morphogenetic proteins (BMPs), insulin-like growth factors (IGFs), basic fibroblast growth factor (bFGF), cartilage derived morphogenetic protein (CDMP), growth and differentiation factors (GDFs), LIM mineralization proteins, transforming growth factor beta family (TGF-β), and other bone proteins, such as CD-RAP. These proteins can be recombinantly produced or obtained and purified from an animal that makes the proteins without the use of recombinant DNA technology. Recombinant human BMP is referred to as “rhBMP”; recombinant human GDF is referred to as “rhGDF”. Any bone morphogenetic protein is contemplated, including bone morphogenetic proteins designated as BMP-1 through BMP-18. Mimetics of growth factors can also be used in the devices of the present invention for inducing the growth of bone.

Each BMP may be homodimeric, or may be heterodimeric with other BMPs (e.g., a heterodimer composed of one monomer each of BMP-2 and BMP-6) or with other members of the TGF-β superfamily, such as activins, inhibins and TGF-β 1 (e.g., a heterodimer composed of one monomer each of a BMP and a related member of the TGF-β superfamily). Any of these substances may be used individually or in mixtures of two or more. One or more statins may also be included in a therapeutic substance. Non-limiting examples of statins that may be included in the devices of the present invention include atorvastatin, cerivastatin, fluvastatin, lovastatin, mavastatin, pitavastatin, pravastatin, rosuvastatin and simvastatin. The therapeutic substance may include various other organic species known to induce bone formation, and combinations thereof.

Embodiments of the sheath may include two or more layers with differing therapeutic characteristics. By way of non-limiting example, a top layer may include an antibiotic to prevent postoperative infection; a second layer may include a chemotherapy agent to prevent recurrence of a tumor; a third layer may include a bioresorbable non-therapeutic to allow the effects of the chemotherapy agent to dissipate before introduction of the next therapeutic agent; and a fourth layer may include a bone growth promoting substance. Any other effective combination of layers may be used with one or more therapeutic substances and may or may not include inert layers, such as a bioresorbable non-therapeutic layer.

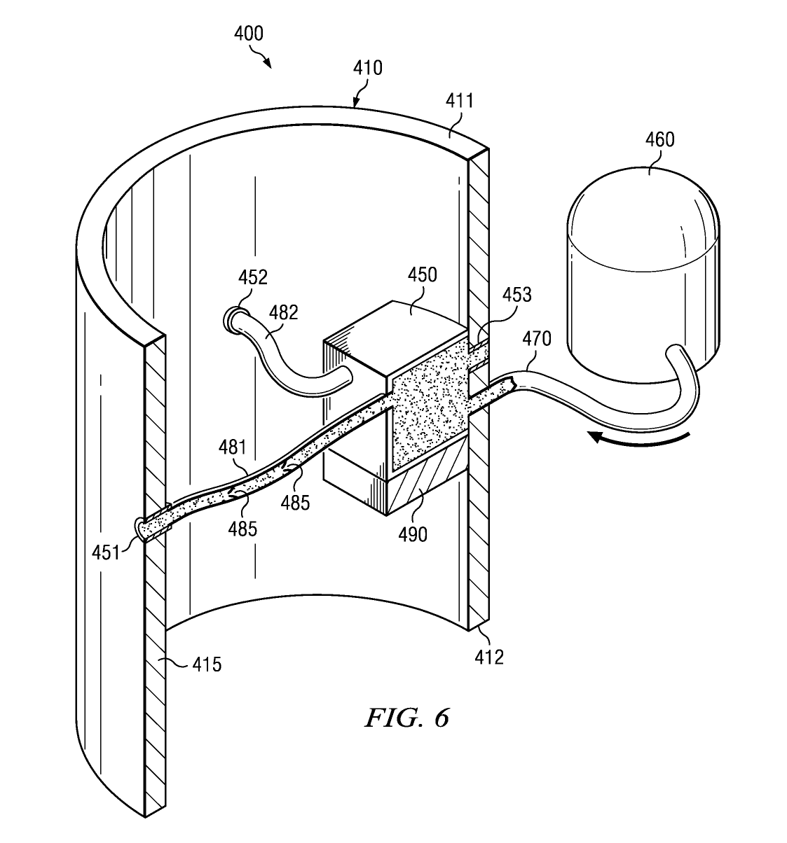

A medical implant 400 configured to deliver a therapeutic substance is illustrated in FIG. 6. The illustrated medical implant 400 includes a vertebral body replacement device 410. The vertebral body replacement device 410 shown has a first end 411 and a substantially opposite second end 412. A side 415 of the vertebral body replacement device 410 extends from the first end 411 to the second end 412. The side 415 defines an interior of the vertebral body replacement device 410. The interior is said to be defined by the side 415 in that the side 415 approximately lays out a boundary of the interior of the vertebral body replacement device 410. In other embodiments, two or more sides may be joined together in the formation of a vertebral body replacement device.

The medical implant 400 shown includes a therapeutic substance reservoir 450 located within an interior of the vertebral body replacement device 410. In other embodiments, a therapeutic substance reservoir is located outside of the interior of a vertebral body replacement device. For example and without limitation, a therapeutic substance reservoir may be located outside of the interior of a vertebral body replacement device, but within a patient's body. In other embodiments, a therapeutic substance reservoir may be located outside of the interior of a vertebral body replacement device, and outside of a patient's body. As shown in FIG. 6, there is a second therapeutic substance reservoir 460. The second therapeutic substance reservoir 460 may be located inside or outside of a patient's body. The second therapeutic substance reservoir 460 in the illustrated embodiment may serve as a supply or re-supply mechanism for the therapeutic substance reservoir 450 via a conduit 470. More than one therapeutic substance reservoir may be employed with some embodiments of the medical implant. The more than one therapeutic substance reservoir may be located together or separately at any effective location.

The medical implant 400 also includes nozzles 451, 452, 453 in fluid communication with the therapeutic substance reservoir 450. Any number of nozzles may be present with other embodiments. The nozzles 451, 452, 453 are coupled to the vertebral body replacement device 410 to direct therapeutic substance from the interior or the vertebral body replacement device 410. Nozzles may be located anywhere along a medical implant to direct therapeutic substance in a clinically advantageous way. The nozzles 451, 452 are shown in FIG. 6 in fluid communication with the therapeutic substance reservoir 450 through respective tubes 481, 482. The nozzle 453 is illustrated directly connected to the therapeutic substance reservoir 450 to provide fluid communication. Other embodiments may include any number or type of fluid communication mechanisms. The nozzles 451, 452, 453 are directed from the interior of the vertebral body replacement device 410 substantially transversely to the side 415. In other embodiments, nozzles may be directed from the interior in any clinically effective direction.

The medical implant 400 may also include one or more valves to control flow of therapeutic substance from the nozzles 451, 452, 453. Valves may be located at a nozzle, along any of the respective tubes, at the therapeutic substance reservoir, outside of the medical implant, or at any other effective location. Valves may be controlled by any effective signal mechanism, including but not limited to, electrical, radio frequency, and pressure. Valves may be signaled directly or through one or more controller devices. By way of non-limiting example in FIG. 6, one or more signal wires may be integrated with the conduit 470 to carry signals into the medical device 400. The signal wires may be connected to a controller 490. The controller 490 may be configured to generate an increased pressure within the therapeutic substance reservoir 450, or elsewhere in the device, or to open and close valves within the therapeutic substance reservoir 450, at the nozzles 451, 452, 453, and along tubes 481, 482. Embodiments of the tubes and nozzles may also include passive, one-way valves to control the direction of flow therapeutic substance. Example one-way valves 485 are shown in the tube 481. Any effective combination of controlled and passive valves is contemplated. Pressure or flow within embodiments of a therapeutic substance reservoir, nozzles, and tubes may be generated by any mechanism. Example mechanisms include but are not limited to, pistons, bellows, piezoelectric driven pumps, and bubble jet spray mechanisms similar to mechanisms used with printing devices. By way of non-limiting example, nozzles may be directed towards a region of a spine or surrounding area from which a tumor has been removed or where a recurrence of a tumor has been detected.

In some embodiments, no nozzles are directed from the interior of a vertebral body replacement device through certain, designated portions of the vertebral body replacement device. In these embodiments, the designated portions may be configured to be implanted adjacent to a spinal cord. This configuration may be useful to avoid directing therapeutic substances toward a spinal cord. Similarly, in some embodiments such a configuration may be used to avoid directing therapeutic substances toward vascular structures, such as, for example, the great vessels along an anterior portion of a spinal column. Any other anatomical part, including anatomical parts sensitive to a therapeutic, may be effectively avoided by selective direction of a therapeutic substance.

In some embodiments, two or more therapeutic substances may be directed through different nozzles of the medical implant. Two or more therapeutic substances may be directed through the same nozzle at different times or as a mixture. The medical implant 400 illustrated in FIG. 6, and other similar devices, may include any of the therapeutic substances disclosed herein in any composition, form, or mixture.

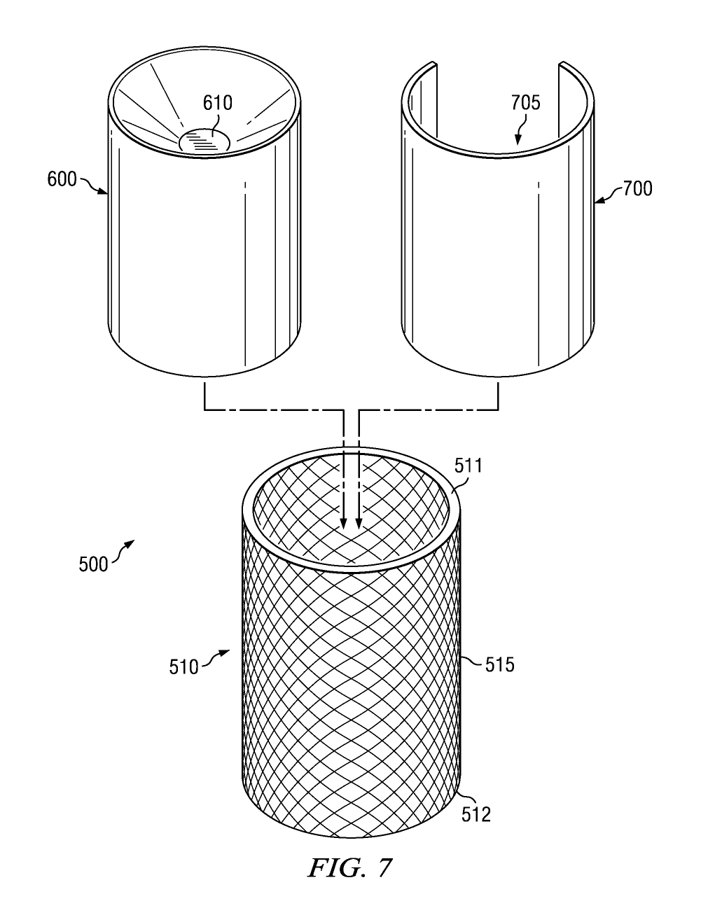

A medical implant 500 configured to deliver a therapeutic substance is illustrated in FIG. 7. The medical implant 500 shown includes a vertebral body replacement device 510 having a first end 511, a substantially opposite second end 512, and a side 515 extending from the first end 511 to the second end 512. The side 515 at least in part defines an interior of the vertebral body replacement device 510. The illustrated medical implant 500 includes an insert 600, 700. As shown, either of alternative inserts 600, 700 may be placed inside the vertebral body replacement device 510. Note that the nomenclature “insert” does not mean that either insert 600, 700, or another insert of the medical implant 500, needs to be completely placed within the vertebral body replacement device 510.

Either of the inserts 600, 700 may be captured within the interior of the vertebral body replacement device 510, as indicated by the arrows in FIG. 7. The inserts 600, 700 are separate, modular devices that are combined with the vertebral body replacement device 510, but in other embodiments, similar components may be, in whole or in part, integrated with a vertebral body replacement device. Inserts of various embodiments include one or more therapeutic substances deliverable from the inserts. The therapeutic substances of the medical implant 500 may include any of the therapeutic substances disclosed herein in any effective composition, form, or mixture. The therapeutic substances of the medical implant 500 may be delivered in any direction, in any amount, and at any rate that is effective.

The insert 600 is shown in cross-sectional view in FIG. 8. A center chamber 625 of the insert 600 is illustrated containing one or more therapeutic substances. The center chamber 625 is plugged by biodegradable material 610 such that the biodegradable material 610 acts as a release mechanism for permitting the one or more therapeutic substances to be delivered from the insert 600. The biodegradable material 610 is configured to dissolve after implantation of the medical implant 500 in a patient. Once at least one of the portions of biodegradable material 610 has dissolved to provide an open passage into the center chamber 625, the one or more therapeutic substances may be delivered from the insert 600. In the illustrated configuration, therapeutic substance may be delivered primarily toward the first and second ends 511, 512 of the vertebral body replacement device 510. However, in other embodiments, therapeutic substance may be delivered in any desired direction by reconfiguration of openings and biodegradable materials.

An insert 650, which may also be captured within the interior of the vertebral body replacement device 510, is shown in cross-sectional view in FIG. 9. A first chamber 651, a second chamber 652, and a third chamber 653 of the insert 600 are illustrated containing one or more therapeutic substances. The therapeutic substance in each of the chambers 651, 652, 653 may be the same or may be different, and may be any of the therapeutic substances disclosed herein. The chambers 651, 652, 653 are bounded by four portions of biodegradable material 661, 662, 663, 664, respectively. The chambers 651, 652, 653 are plugged by the biodegradable material 661, 662, 663, 664 such that the biodegradable material acts as a release mechanism for permitting the one or more therapeutic substances to be delivered from the insert 600. The biodegradable material 661, 662, 663, 664 may be configured to dissolve after implantation of the medical implant 500 in a patient. For the illustrated embodiment, therapeutic substance in the first chamber 651, for example, may be reached in the first chamber 651 by an aqueous substance from a patient's body when the first portion of biodegradable material 661 is adequately dissolved. When aqueous substance is allowed into the first chamber 651, then the aqueous substance is able to disperse and deliver therapeutic substance and to contact the second portion of biodegradable material 662. A similar progression through the fourth portion of biodegradable material 664, the third chamber 653, and the third portion of biodegradable material 663 may occur from the opposite end of the insert 650. The second chamber 652 may be reached from either end of the insert 650 following dissolving of the portions of biodegradable material and therapeutic substances between the second chamber 652 and the respective ends. Any desired number of bioresorbable material portions, chambers, and therapeutic substances may be employed in various embodiments. In the illustrated configuration, therapeutic substance may be delivered primarily toward the first and second ends 511, 512 of the vertebral body replacement device 510. However, in other embodiments, therapeutic substance may be delivered in any desired direction by reconfiguration of plugged open areas and biodegradable materials.

The insert 700 that may also be captured within the interior of the vertebral body replacement device 510, as shown in FIG. 7. The insert 700 may be substantially flat until bent to be placed within the vertebral body replacement device 510. Alternatively, the insert 700 may be pre-formed to the shape of the interior of the vertebral body replacement device 510. The illustrated insert 700 has a circumference that is less than the circumference of the vertebral body replacement device 510 such that a portion of the circumference of the vertebral body replacement device 510 does not include contact with the insert 700, and consequently, any therapeutic substance associated with the insert 700. This portion of the vertebral body replacement device 510 may be placed in an area where delivery of therapeutic substance is not desired, such as near a patient's spinal cord. Some embodiments of an insert of this form may have a circumference that is greater than the circumference of the vertebral body replacement device 510 such that a part of the insert would overlap when in place in the vertebral body replacement device 510. The insert 700 also includes a cannula 705 suitable for receiving bone growth promoting substance or other material. In other embodiments, a cannula may be offset from the center of an insert or an insert may contain more than one cannula. The insert 700 may include multiple layers with different therapeutic and non-therapeutic substances, as further described herein with regard to multiple layer sheaths.

In another embodiment, an insert of the medical implant 500 may be a device that controls release of a therapeutic substance in any of the ways disclosed herein in association with the medical implant 400. In such an embodiment, the entire medical implant 400, or similar construct, may be placed within the vertebral body replacement device 510 to assemble a completed medical implant 500.

Terms such as around, near, opposite, top, side and the like have been used herein to note relative positions. However, such terms are not limited to specific coordinate orientations, but are used to describe relative positions referencing particular embodiments. Such terms are not generally limiting to the scope of the claims made herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A medical implant configured to deliver a therapeutic substance comprising:
a vertebral body replacement device having a first end and a substantially opposite second end; and
a sheath coupled to the vertebral body replacement device and covering substantially all of the vertebral body replacement device from the first end to the second end, wherein the sheath includes therapeutic substance deliverable from the sheath to one or more anatomic structures positionable adjacent to the medical implant.

2. The medical implant of claim 1 wherein the vertebral body replacement device is an expandable device configured to be expanded from a first state where the first end the and the second end are a distance apart to a second state where the first end and the second end are a greater distance apart.

3. The medical implant of claim 2 wherein the sheath includes an elastic material such that the sheath will expand with the vertebral body replacement device to cover substantially all of the vertebral body replacement device in the first state and in the second state.

4. The medical implant of claim 1 wherein the sheath is at least coupled to the vertebral body replacement device at a first location near the first end of the vertebral body replacement device and at a second location near the second end of the vertebral body replacement device.

5. The medical implant of claim 1 wherein the therapeutic substance contains one or more of the following substances: antibiotic, antiseptic, analgesic, anti-inflammatant, anti-coagulant, antifungal, steroid, enzyme, immunosuppressant, antithrombogenic composition, vaccine, hormone, growth inhibitor, growth stimulator, chemotherapy drugs, biodegradable non-therapeutic, and bone growth promoting.

6. The medical implant of claim 1 wherein the sheath includes two or more layers with differing therapeutic characteristics.

7. The medical implant of claim 1 wherein the sheath includes a region containing therapeutic substance and a separate region containing no therapeutic substance.

8. A medical implant configured to deliver a therapeutic substance comprising:
at least one therapeutic substance reservoir;
two or more nozzles in fluid communication with the at least one therapeutic substance reservoir; and
a vertebral body replacement device having a first end, a substantially opposite second end, and one or more sides extending from the first end to the second end, wherein the one or more sides at least in part define an interior of the vertebral body replacement device;
wherein the two or more nozzles are coupled to the vertebral body replacement device to direct therapeutic substance from the interior of the vertebral body replacement device.

9. The medical implant of claim 8 wherein the therapeutic substance reservoir is within the interior of the vertebral body replacement device.

10. The medical implant of claim 8 wherein the therapeutic substance reservoir is outside of the interior of the vertebral body replacement device.

11. The medical implant of claim 8, further comprising one or more valves to control flow of therapeutic substance from the two or more nozzles.

12. The medical implant of claim 11, further comprising a controller that operates the one or more valves.

13. The medical implant of claim 8 wherein at least one of the two or more nozzles is directed from the interior of the vertebral body replacement device through the one or more sides and at least one of the two or more nozzles is directed from the interior of the vertebral body replacement device substantially transversely to the one or more sides.

14. The medical implant of claim 8 wherein none of the two or more nozzles is directed from the interior of the vertebral body replacement device through a designated portion of the vertebral body replacement device, wherein the designated portion is configured to be implanted adjacent to a sensitive anatomical structure.

15. The medical implant of claim 8 wherein two or more therapeutic substances are directed through the two or more nozzles.

16. A medical implant configured to deliver a therapeutic substance comprising:
a vertebral body replacement device having a first end, a substantially opposite second end, and one or more sides extending from the first end to the second end, wherein the one or more sides at least in part define an interior of the vertebral body replacement device; and
an insert captured within the interior of the vertebral body replacement device, wherein the insert includes one or more therapeutic substances deliverable from the insert, and wherein the insert includes a release mechanism for permitting the one or more therapeutic substances to be delivered from the insert.

17. The medical implant of claim 16 wherein the insert includes a cannula suitable for receiving bone growth promoting substance.

18. The medical implant of claim 16 wherein the release mechanism includes one or more bioresorbable plugs that are configured to dissolve after implantation and allow one or more therapeutic substances to be delivered from the insert.

19. The medical implant of claim 18 wherein a first chamber containing at least one therapeutic substance is at least in part bounded by a first bioresorbable plug and a second bioresorbable plug, and wherein after the first bioresorbable plug is dissolved, aqueous substance is allowed into the first chamber to contact the second bioresorbable plug in a position to dissolve the second bioresorbable plug.

20. The medical implant of claim 16 wherein the release mechanism includes one or more valves that are configured to control flow of one or more therapeutic substances from the two or more nozzles.
